(19) United States
(12) Patent Application Publication
(10) Pub. No.: US 2013/0060334 A1
(43) Pub. Date: Mar. 7, 2013

(54) MENISCUS REPAIR ASSEMBLY AND METHOD

(71) Applicant: Peter R. Kurzweil, Long Beach, CA (US)

(72) Inventor: Peter R. Kurzweil, Long Beach, CA (US)

(73) Assignee: ORTEQ B.V., GX Groningen (NL)

(21) Appl. No.: 13/581,120

(22) PCT Filed: Feb. 23, 2011
(86) PCT No.: PCT/US2011/025852

§ 371 Date: Nov. 12, 2012

Related U.S. Application Data
(60) Provisional application No. 61/307,933, filed on Feb. 25, 2010.

(51) Int. Cl. A61F 2/08

(52) U.S. Cl. 623/14.12

(57) ABSTRACT

The present invention provides an assembly for repairing a tear or lesion in a body tissue such as a meniscus including a scaffold material and methods using such an assembly in repairing a tear or lesion.

### CROSS REFERENCE TO RELATED APPLICATIONS

The present invention claims the benefit of the following United States Provisional Patent Application Nos. 61/307,933, filed Feb. 25, 2010. The contents of this application is incorporated herein by reference.

### FIELD

The present invention relates generally to an assembly and method for use in repairing a tear or lesion in a meniscus during arthroscopic surgery.

### BACKGROUND

The meniscus is a fibrocartilaginous structure in the knee joint which performs multiple critical functions, including contributing to normal knee biomechanics and the general well-being of the joint. Generally, the menisci are comprised of two C-shaped fibrocartilaginous structures residing on the tibial plateau. The peripheral rim of a meniscus is thick, tapering to a thin, free inner border. The superior surface is concave to contact the femoral condyles, while the inferior surface is flat to contact the tibial plateau. The fibers forming the menisci are mainly oriented circumferentially throughout the meniscus, parallel to the peripheral border, to withstand hoop stresses placed upon the meniscus by the femoral condyles.

A peripheral region or zone of the meniscus is generally referred to as a red/red zone that has good blood supply. A central region or zone of the meniscus is generally referred to as a white/white zone that is avascular. An intermediate region or zone is generally referred to as red/white zone that has variable blood supply. It is generally recognized that repair of meniscal lesions or tears to the extent possible, is preferable to excision so as to attempt to maintain the normality of the meniscus and have it continue to function as intended. In addition, it is important to maintain vascularity within the peripheral area and intermediate area of the meniscus to promote healing.

There are many techniques employed to repair damaged soft tissue such as the meniscus. These techniques include suturing, stapling, taping and the like. Selection of which technique to employ depends upon the type of soft tissue being repaired, the soft tissue location and the required strength of the repair. While there exist many techniques to repair soft tissue, there is a growing need to easily and quickly repair a tear or lesion in a meniscus in the knee during arthroscopic surgery. Tissue engineering techniques have been developed to provide alternative strategies to repair such tissues as a torn meniscus. Several tissue engineering strategies have the potential to restore or preserve function to torn menisci. These include all biological repair techniques, techniques to enhance the ability to repairs in the avascular zone, and scaffolds to replace excised portions of the meniscus. Scaffolds may provide a mechanism for tissue regeneration and cellular repopulation of otherwise irreparable menisci thereby preserving meniscus function in knees treated with excision.

The use of such scaffolds involves removing a significant amount of meniscal tissue to provide adequate space for the scaffold implant. Excision up to the red/red zone or red/white zone is often needed to provide adequate blood supply for the regeneration and repair of the excised/damaged tissue. Frequently, such use of scaffolds thus includes not only removing damaged tissue but also the removal of healthy tissue. In addition, the implantation of scaffolds requires the relatively complicated procedure of shaping the meniscal defect, then shaping a piece of material to match the defect (or excised material), followed by inserting this shaped implant material into the knee and securing it in place by, for example, suturing.

Such use of scaffolds in repairing meniscal tissue may be complicated and may involve compromising meniscal integrity during the healing process. Meniscal integrity, however, may be the key factor in the long-term outcomes of reconstruction and repair. Patients undergoing partial meniscectomy appear to experience more pain and degenerative radiographic changes than patients undergoing meniscus repair. A higher acceptance by surgeons and patient may be achieved using a relatively easy and less invasive procedure to repair a tear or lesion in a meniscus which preserves meniscal integrity. Thus to improve the potential healing response of meniscal tears and expand the indications of “repairable” menisci, alternative repair techniques are needed. Improvements in tissue engineering and surgical techniques with minimal tissue damage and reduced pain associated with tissue repair, aimed at preserving meniscal function, may provide significant benefits in the potential healing response of meniscal tears. The assembly and method of the present invention provides for meniscus repair while preserving meniscal integrity.

### SUMMARY

An assembly for repairing a tear or lesion in a meniscus of the knee comprising: a) a pair of opposing surfaces, a superior surface and an inferior surface, each having an inner side and an outer side, wherein the opposing surfaces are joined at least along one edge, and b) a scaffold material implant extending from the joining edge towards the opposing non-joined edge of the opposing surfaces, wherein said scaffold material is adapted to allow re-growth of the meniscus tissue while providing support to retain meniscal function.

An assembly for repairing a tear or lesion in a meniscus of the knee comprising: a pair of opposing surfaces, a superior surface and an inferior surface, each having an inner side and an outer side, wherein the opposing surfaces are joined at least along one edge, wherein the inner side of at least one opposing surface comprises a scaffold material adapted to allow re-growth of the meniscus tissue while the assembly provides support to retain meniscal function. Alternatively, the assembly comprises a single surface of which at least the inner side comprises a scaffold material.

According to various features, the opposing surfaces in the assemblies for repairing a tear or lesion in a meniscus of the knee comprise a thin sheet of scaffold material.

According to other features, the scaffold material may be a porous structure such as for example comprising natural or synthetic fibers in a fabric or non-woven film material, or comprising polymer foam material. This scaffold material is preferably degradable and/or biocompatible. Preferably the scaffold material comprises a degradable and biocompatible polymer foam.

A method for repairing a tear or lesion in the meniscus of the knee includes forming a passage in the knee to repair the tear or lesion, the passage defining an entrance and an exit. The assembly is passed through the passage. The assembly is manipulated whereby the opposing surfaces cover the damaged portion of the meniscus. Further, where the assembly includes a scaffold material implant, the assembly is manipulated to cover the lesion in the meniscus. The assembly can then secured to the meniscal tissue using a securing means.

Further areas of applicability of the present disclosure will become apparent from the detailed description provided hereinafter. It should be understood that the detailed description and various examples, while indicating various embodiments of the invention, are intended for purposes of illustration only and are not intended to limit the scope of the following claims.

### BRIEF DESCRIPTION OF THE FIGURES

FIG. 1: Shows a diagram representing meniscus repair of a meniscus having a tear or lesion (A) wherein a substantial portion of the meniscus is excised (B) to provide space for an implant of scaffold material which has been shaped in to match the excised portion (C) after which the shaped scaffold material is implanted in the excised portion of the meniscus (D).

FIG. 2: Shows a perspective view of a meniscus repair assembly according to the present invention comprising a scaffold material implant (FIG. 2A), and a side view of the same meniscus repair assembly.

FIG. 3: Shows top view of a meniscus repaired using the meniscus repair assembly according to the present invention providing a scaffold material implant at the tear or lesion of the meniscus.

FIG. 4: Shows a perspective view of a meniscus repaired with an thin sheet of the assembly according to the present invention.

FIG. 5: Shows a side view of a meniscus repaired with a thin folded sheet of the assembly according to the present invention which is being secured to meniscal tissue.

FIG. 6: Shows a side view of a meniscus repaired with a thin sheet of the assembly according to the present invention which is being secured to meniscal tissue.

### DETAILED DESCRIPTION OF VARIOUS EMBODIMENTS

The following description of various embodiments is merely exemplary in nature and is not intended to limit the application or uses.

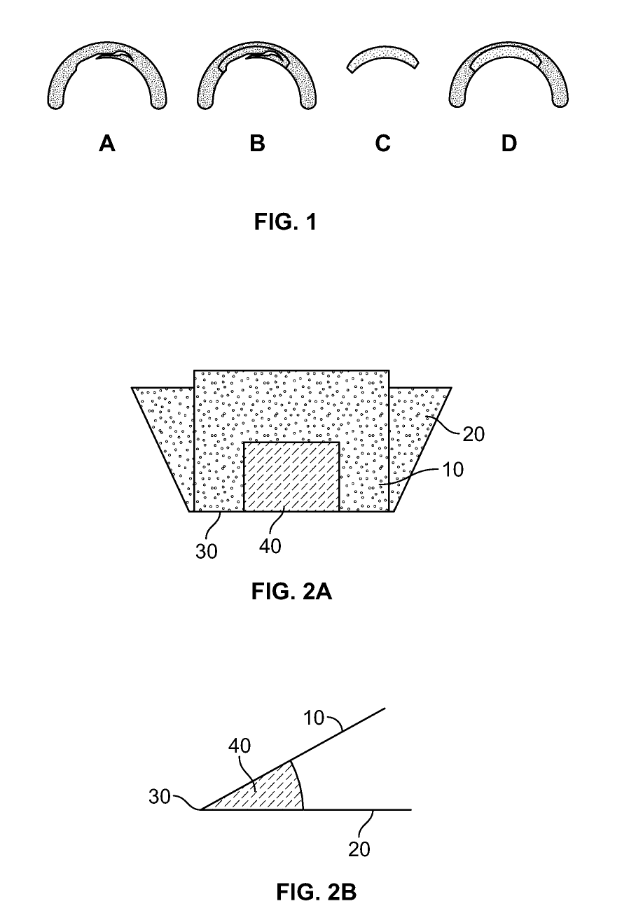

In order to repair more difficult to repair tears or lesions of the meniscus or those menisci with more extensive damage, a technique may be used wherein a scaffold material is implanted in the injured meniscus. This process of meniscus repair as is illustrated in FIG. 1 includes identifying a tear or lesion in a meniscus (A). Subsequently a substantial portion of the meniscus is excised (B) to provide space for an implant of scaffold material, which excision extends to the red/red zone or red/white zone of the meniscus to provide sufficient blood supply for healing. The implant of scaffold material needs to be shaped (C) to match the excised portion of the meniscus. This shaped implant of scaffold material (C) is then implanted in the excised portion of the meniscus (D). Such use of scaffolds in repairing meniscal tissue may be complicated and may involve compromising meniscal integrity during the healing process. Meniscal integrity however may be the key facto in the long-term outcomes of reconstruction and repair. Improvements in tissue engineering and surgical techniques with minimal tissue damage and reduced pain associated with tissue repair, aimed at preserving meniscal function may provide significant benefits in the potential healing response of meniscal tears. The assembly and method of the present invention provides for meniscus repair while preserving meniscal integrity. The assembly of the present invention may promote healing of a lesion in the meniscus by stabilizing the lesion more than using sutures alone could accomplish and it provides a healing scaffold across which healing can occur, bridging the tear or lesion in the meniscus.

With initial reference to FIGS. 2A and 2B, an assembly for repairing a tear or lesion in a meniscal tissue is shown. The assembly according to the present invention comprises a pair of opposing surfaces, a superior surface 10 and an inferior surface 20. Each surface having an inner side and on outer side. The opposing superior surface and inferior surfaces are joined at least along one edge 30 from which a scaffold material implant 40 extends towards the opposing non joining edge of the superior 10 and/or inferior surfaces 20. The scaffold material may extend entirely or partially to toward the edge of the opposing surfaces 10 and 20 as is needed to repair a lesion in the meniscus tissue.

The scaffold material 40 provides a matrix which allows re-growth of the meniscal tissue in the scaffold material 40. Further, to promote re-growth of the tissue the scaffold material 40 and/or the oppossing surfaces 10 and 20 may contain additional growth promoting materials such as for example blood clot, bone marrow, platelet rich plasma (PRP), and growth factors. The scaffold material 40 provides support to the meniscus tissue and enables the meniscus to retain its function while the lesion heals. The opposing surfaces 10 and 20 provide additional support to cover the tear or lesion in the meniscus without the need for extensive removal by excision of meniscal tissue to provide space for implant material of which one edge needs to be in proximity to the red/red zone or red/white zone to allow sufficient blood supply for re-growth of the damaged tissue. The superior 10 and/or inferior 20 surfaces of the assembly according to the present invention provide close proximity to the red/red zone or red/white zone with sufficient blood supply to allow re-growth into the scaffold material 40.

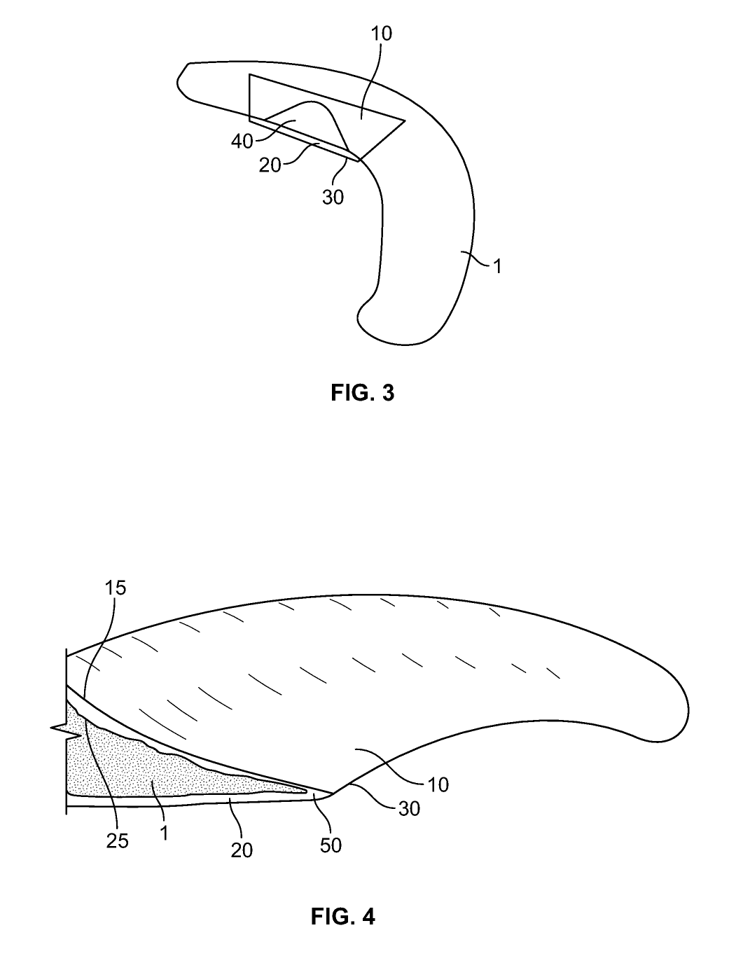

FIG. 3 shows an oblique view of the assembly according to the present invention covering a lesion of an damaged meniscus 1. The opposing superior 10 and inferior 20 surfaces are joined together along edge 30 to support a scaffold material 40 that is implanted into the lesion in the meniscus. In order to allow the re-growth of the damaged meniscus 1 the superior 10 and inferior 20 surfaces extend to cover the red/red or red/white zone when the assembly is placed over the lesion in the meniscus. The composition of the superior 10 and inferior 20 surfaces of the assembly is preferably the same as the composition of the scaffold material 40 or alternatively comprises a non-porous film material when the scaffold material 40 extends up to the red/red zone or red/white zone. The composition of the scaffold material 40 can be any porous scaffold material such as for example comprising natural or synthetic fibers in a fabric or non-woven material, or comprising polymer foam material. This scaffold material is preferably degradable and/or biocompatible. Preferably the scaffold material comprises a biocompatible foam that is degradable. Further, both the superior surface 10 and inferior surface 20 can comprise a thin sheet of the scaffold material that allows re-growth of meniscal tissue. Preferably, the thin sheet is pliable so as to smoothly fit over the tear or lesion in the meniscus while providing support for retaining meniscal function. The thin sheet has a strength sufficient to hold sutures and the assembly in a stable manner to better promote healing of the meniscal lesion.

In an alternative embodiment as shown in FIG. 4, the assembly according to the present invention comprises a pair of opposing surfaces, a superior surface 10 and an inferior surface 20. Each surface having an outer side 15 and an inner side 25. The opposing superior surface 10 and inferior surface 20 are joined at least along one edge 30. The inner side 25 of at least one opposing surface comprises a scaffold material that allows re-growth of the meniscal tissue 1 into the tear or lesion thereby repairing said tear or lesion in the meniscus. Further, the inner side 25 may contain additional growth promoting materials such as for example blood clot, bone marrow, platelet rich plasma (PRP), and growth factors. The assembly according to the present invention may be in the form of a folded sheet 50 that covers a substantial part of the meniscus having a tear or lesion. The folded sheet 50 and superior surface 10 and inferior surface 20 can in their entirety comprise a thin sheet of the scaffold material that allows re-growth of meniscal tissue. Preferably, the thin sheet is pliable so as to smoothly fit over the tear or lesion in the meniscus while providing support for retaining meniscal function. The thin sheet has a strength sufficient to hold sutures and the assembly in a stable manner to better promote healing of the meniscal lesion. In order to allow the re-growth of the damaged meniscus 1 the superior 10 and inferior 20 surfaces or folded sheet 50 extend to cover the red/red or red/white zone when the assembly is placed over the lesion in the meniscus. The composition of the superior 10 and inferior 20 surfaces or folded sheet 50 can be any porous scaffold material such as for example comprising natural or synthetic fibers in a fabric or non-woven material, or comprising polymer foam material. This composition is preferably degradable and/or biocompatible. Preferably the composition comprises a biocompatible foam that is degradable. Alternatively, the composition of superior surface 10 and inferior surface 20 or folded sheet 50 may partly comprise a non-porous film material.

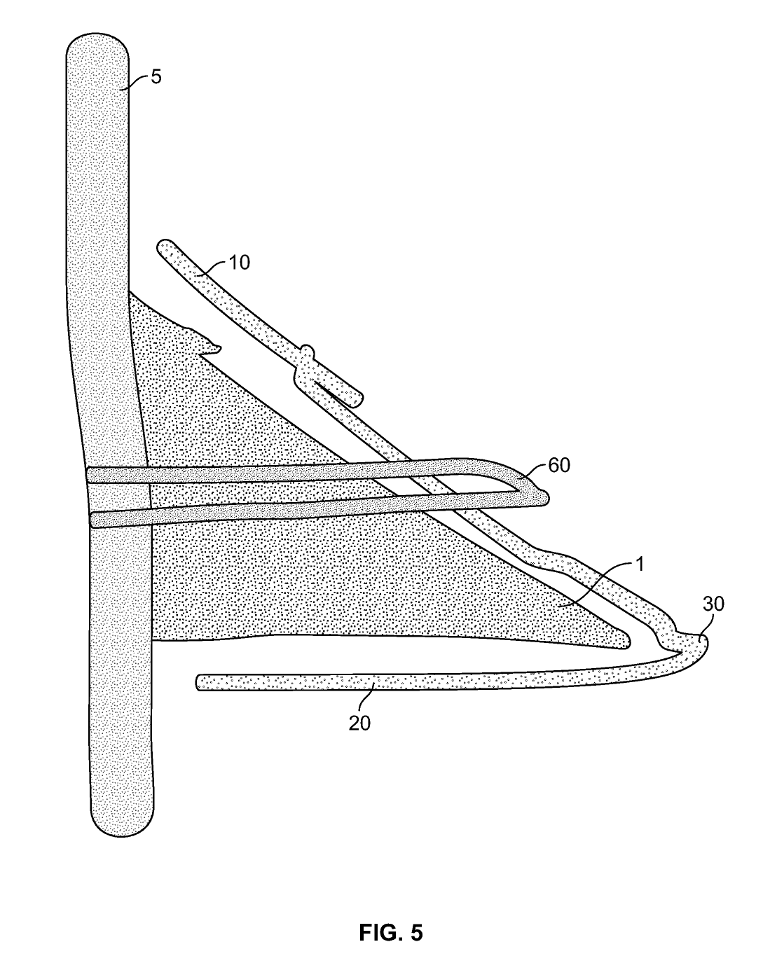

FIG. 5 shows a side view of an assembly according to the invention covering a meniscus 1 for repairing a tear or lesion in the meniscus. The opposing superior 10 and inferior 20 surfaces cover the damaged portion of the meniscus 1 while being joined at edge 30 so as to provide a pocket over the meniscus on both the top section and the bottom section of the meniscus. Any of the above described assemblies according to the invention can be held in place by a securing means 60 which secures the assembly to tissue. The securing means may attach to a capsule 5. As in FIGS. 4 and 5 the opposing superior surface 10 and inferior surface 20 together with the joining edge 30 may form a folded sheet 50 as the assembly which can be placed over the meniscus to repair the tear or lesion.

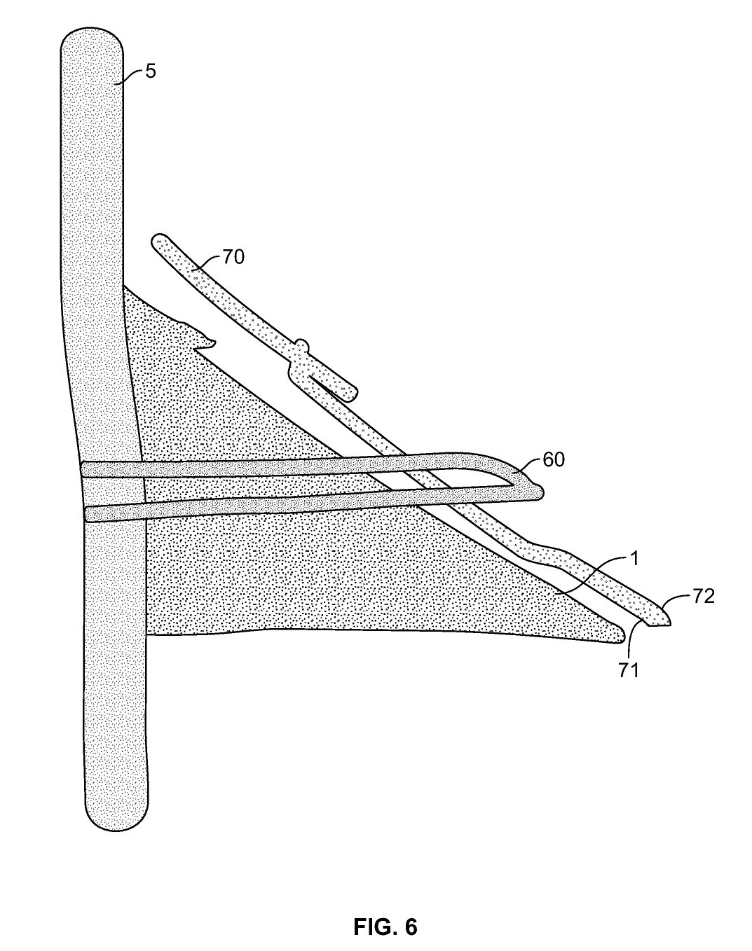

In an alternative embodiment as shown in FIG. 6 the assembly according to the invention comprises one surface 70, having an inner side 71 and an outer side 72. The inner side 71 of the surface 70 comprises a scaffold material that allows re-growth of the meniscal tissue 1 into the tear or lesion thereby repairing said tear or lesion in the meniscus. Further, the inner side 71 may contain additional growth promoting materials such as for example blood clot, bone marrow, platelet rich plasma (PRP), and growth factors. Preferably, the entire surface 70 consist of the scaffold material. This surface 70 is preferably a thin sheet of the scaffold material. Preferably, the thin sheet is pliable so as to smoothly fit over the tear or lesion in the meniscus while providing support for retaining meniscal function. This thin sheet of the surface 70 preferably extends over the tear or lesion and the red/red zone or red/white zone of the meniscus to enable sufficient blood supply for re-growth of the meniscal tissue into the tear or lesion. The assembly comprising surface 70 can be secured over the tear or lesion of the meniscus using a securing means 60. The securing means may attach to a capsule 5.

The composition of the scaffold material that is either used for the scaffold material implant or for any of the inner sides or surfaces of the assembly according to the invention can be any porous scaffold material such as for example comprising natural or synthetic fibers in a fabric or non-woven material, or comprising polymer foam material. Additionally, the scaffold material may further comprise additional growth promoting materials such as for example blood clot, bone marrow, platelet rich plasma (PRP), and growth factors. The scaffold material is preferably degradable and/or biocompatible. Preferably the scaffold material comprises a biocompatible polymer foam that is degradable. Such foams for use in the assembly according to the present invention have properties especially useful for such assembly, including having a modulus of compression between about 50 kPa to about 1500 kPa, preferably about 250 kPa to about 400 kPa, a tear strength of greater than or equal to about 3 N/mm, and flexibility (strain at break) of about 100% or higher. These advantageous properties are in part due to the high molecular weight of the polymers in the foam and the in part due to the interconnectivity of the polymers in the foam. This high molecular weight and interconnectivity are achieved by the process of making the polyurethane polymer and by the process of making the foam from the polyurethane polymer as described for example in International Patent Application No. PCT/IB2009/005958, filed May 19, 2009. The final average molecular weight of the polymer in the foam is preferably about 110 kg/mol to about 240 kg/mol. More preferably the average molecular weight of the polymer is about 120 kg/mol to about 240 kg/mol. Even more preferably, the average molecular weight of the polymer in the foam is 140 kg/mol to about 240 kg/mol.

The polymer in such foam my be a polyurethane prepared by a process comprising the steps of: (a) reacting a diol, preferably a C₁-C₁₀ alkyl diol, more preferably 1,4-butanediol, with an oxygen containing compound that can form a macrodiol by ring-opening polymerization, preferably a lactone, more preferably ε-caprolactone, to provide a macrodiol, wherein the reaction is carried out to completion, preferably until the unreacted remaining oxygen containing compound that can form a macrodiol by ring-opening polymerization is less than 0.5% by mole equivalents of the total amount of the oxygen containing compound, more preferably less than about 0.2% by mole equivalents; (b) treating the macrodiol with a diisocyanate, to obtain a macrodiisocyanate, wherein the unreacted diisocyanate is removed under a pressure of less than about 0.01 mbar, preferably less than about 0.003 mbar, preferably until the remaining amount of unreacted diisocyanate is between −5% to 5% by mole equivalent of the calculated required amount of diisocyanate in the reaction, more preferably between −2% and 2% by mole equivalents, even more preferably between −1% and 1% by mole equivalent; most preferably between −0.5% and 0.5% by mole equivalents; and (c) reacting the macrodiisocyanate with a diol chain extender, preferably a diol, more preferably a C₁-C₁₀ alkyl diol, even more preferably 1,4-butanediol, wherein the molar ratio of macrodiisocyanate:diol is 1.00:1.00 to 1.00:1.09, preferably 1.00:1.01 to 1.00:1.03.

A biocompatible foam that is degradable as may be used in the assembly according to the present invention may be prepared from such polyurethane by for example a process comprising: (a) preparing a solution of about 20% to about 50% (w/v), preferably of about 30% to about 45% (w/v), preferably about 36% (w/v) of polyurethane in an appropriate solvent, preferably wherein the polyurethane is soluble, preferably DMSO, DMF, chloroform, 1,4-dioxane, NMP, m-cresol, dimethyl acetamide, more preferably DMSO; (b) combining the solution with a non-solvent, preferably water or a C₁-C₆ alkyl diol, more preferably water, to obtain a solution, preferably the amount of non-solvent added to the solution is in an amount from 5% to 30% (v/v), more preferably 5% to 20%, most preferably from 5% to 10% (v/v); (c) adding a pore forming material not soluble in the solvent, preferably a salt, more preferably an alkali metal or alkaline earth metal salt, even more preferably an halogen salt of an alkali metal or alkaline earth metal, most preferably NaCl, to obtain a viscous mixture; (d) pouring the viscous mixture into a mold and cooling, in any order to obtain a molded material; and (e) washing the molded material with a non-solvent wherein the polyurethane polymer is insoluble but wherein the pore forming material can be dissolved to obtain a foam for use in an assembly for repairing a tear or lesion in a meniscus according to the present invention.

Thus the current invention provides a conservative approach to meniscal repair involving meniscal preservation by providing an assembly of at least one surface comprising a scaffold material or including scaffold material implant that allows for re-growth of meniscal tissue into the tear or lesion of the meniscus.

Those skilled in the art can now appreciate from the foregoing description that the broad teachings of the present invention can be implemented in a variety of forms. Further, while some examples illustrate repairing a meniscal tear by securing the assembly to the meniscus using a securing means the securing means may include but are not limited to staples or sutures. Therefore, while this invention has been described in connection with particular examples thereof, the true scope of the invention should not be so limited since other modifications will become apparent to the skilled practitioner upon a study of the drawings, the specification and the following claims.

1. An assembly for repairing a tear or lesion in a meniscus of the knee comprising:
a) a pair of opposing surfaces, a superior surface and an inferior surface, each having an inner side and an outer side, wherein the opposing surfaces are joined at least along one edge, and
b) a scaffold material implant extending from the joining edge towards the opposing non-joined edge of the opposing surfaces,
wherein said scaffold material is adapted to allow re-growth of the meniscus tissue while providing support to retain meniscal function.

2. The assembly of claim 1, wherein the scaffold material implant extends partially towards the non-joining edge so as not to cover the entire inner sides of both the superior and inferior surfaces.

3. The assembly of claim 1, further comprising a means of securing the assembly to tissue.

4. The assembly of claim 1, wherein the composition of the opposing surfaces and the scaffold material are the same.

5. The assembly of claim 1, wherein the scaffold material comprises a biocompatible foam.

6. The assembly of claim 5, wherein the biocompatible foam is degradable.

7. The assembly of claim 6, wherein the biocompatible foam is polyurethane foam.

8. The assembly of claim 1, wherein the opposing surfaces are thin so as to be pliable to smoothly fit over the lesion in the meniscus.

9. An assembly for repairing a tear or lesion in a meniscus of the knee comprising: a pair of opposing surfaces, a superior surface and an inferior surface, each having an inner side and an outer side, wherein the opposing surfaces are joined at least along one edge, and wherein the inner side of at least one opposing surface comprises a scaffold material adapted to allow re-growth of the meniscus tissue while providing support to retain meniscal function.

10. The assembly of claim 9, further comprising a means of securing the assembly to tissue.

11. The assembly of claim 9, wherein the opposing surfaces are composed of the scaffold material.

12. The assembly of claim 9, wherein the scaffold material comprises a biocompatible foam.

13. The assembly of claim 12, wherein the biocompatible foam is degradable.

14. The assembly of claim 13, wherein the biocompatible foam is polyurethane foam.

15. The assembly of claim 9, wherein the opposing surfaces form a folded sheet.

16. The assembly of claim 9, wherein the opposing surfaces are thin so as to be pliable to smoothly fit over the tear or lesion in the meniscus.

17. An assembly for repairing a tear or lesion in a meniscus of the knee comprising: a surface, having an inner side and an outer side, wherein the inner side of the surface comprises a scaffold material adapted to allow re-growth of the meniscus tissue while providing support to retain meniscal function.

18. The assembly of claim 17, further comprising a means of securing the assembly to tissue.

19. The assembly of claim 17, wherein the surface is composed of the scaffold material.

20. The assembly of claim 17, wherein the scaffold material comprises a biocompatible foam.

21. The assembly of claim 20, wherein the biocompatible foam is degradable.

22. The assembly of claim 21, wherein the biocompatible foam is polyurethane foam.

23. The assembly of claim 17, wherein the surface is thin so as to be pliable to smoothly fit over the tear or lesion in the meniscus.

24. A method for meniscal repair augmentation the method comprising:
a) providing an assembly in accordance with claim 1;
b) inserting the assembly over a damaged portion of a meniscus in a patient, wherein the meniscus has a tear or lesion; and
c) securing the assembly in place covering the damaged portion of the meniscus.

25. A method for meniscal repair augmentation the method comprising:
a) providing an assembly in accordance with claim 9;
b) inserting the assembly over a damaged portion of a meniscus in a patient, wherein the meniscus has a tear or lesion; and
c) securing the assembly in place covering the damaged portion of the meniscus.
